(19) United States
(12) Patent Application Publication
(10) Pub. No.: US 2014/0010768 A1
(43) Pub. Date: Jan. 9, 2014

(54) FLAVORING INGREDIENT IN ORAL COMPOSITIONS

(71) Applicants: Timothy CHAMBERS, Battle Creek, MI (US); Naeemah Hall, Kalarnazoo, MI (US); Mark Morris, Eugene, OR (US); Deven Holmgren, Eugene, OR (US); Ross Sheldon, Kalamazoo, MI (US); Joan E. Harvey, Easton, PA (US); Dino C. Ferri, Lake Hiawathe, NJ (US)

(72) Inventors: Timothy CHAMBERS, Battle Creek, MI (US); Naeemah Hall, Kalarnazoo, MI (US); Mark Morris, Eugene, OR (US); Deven Holmgren, Eugene, OR (US); Ross Sheldon, Kalamazoo, MI (US); Joan E. Harvey, Easton, PA (US); Dino C. Ferri, Lake Hiawathe, NJ (US)

(73) Assignee: WILD FLAVORS, INC., Erlanger, KY (US)

(21) Appl. No.: 13/544,052

(22) Filed: Jul. 9, 2012

(51) Int. Cl. A23L 1/222; A61Q 11/00; A61K 8/97

(52) U.S. Cl. CPC A23L 1/222; A61K 8/97; A61Q 11/00
USPC 424/58; 424/49; 426/651; 426/3; 426/631; 426/590; 426/2

(57) ABSTRACT

An oral composition containing an essential oil that provides a physical warming sensation when administered to the oral cavity and a method for achieving a physical warming sensation by administering the oral composition. The essential oil is rich in cis and trans-Piperitone Oxide and Piperitenone Oxide that impart unique organoleptic characteristics when the essential oil is used as the primary flavoring ingredient or as an additive in flavor compositions for chewing gum, confections, oral care applications, beverages, and general food applications.

### FIELD OF THE INVENTION

The present invention relates to an oral composition comprising an essential oil exhibiting unique organoleptic properties for use in oral products and the use of oral compositions containing the essential oil to obtain the unique organoleptic sensation. The essential oil is derived from Mentha longifolia ssp. Hymalaiensis.

### BACKGROUND

Mint plants are cultivated as industrial crops in many countries in order to recover essential oils which are known to contain high amounts of monoterpenes and a full range of oxygenated terpene ingredients. Mentha spicata, commonly referred to as ‘Native Spearmint’, is an important source of flavor in various confectionery and food items due to the presence of l-Carvone as the major component in its essential oil. The spearmint oil and its main constituent, l-Carvone, are used extensively for various preparations ranging from medicine to flavored chewing gums, toothpastes and other food products. The commercially important Mentha spicata predominantly has l-Carvone in its essential oil at levels that exceed 60% of the total composition.

Using commonly available taxonomic keys for the genus Mentha, Mentha longifolia ssp. hymalaiensis would typically be classified as a variety of Mentha spicata since botanical references and many experts on mint taxonomy do not recognize Mentha longifolia as a separate species. As a variety, a clear distinction is drawn for M. longifolia since the plant is native to montane sections of Europe, Asia and Africa and does not occur in a naturalized state in either England and North America. Tucker, A. G., The Big Book of Herbs, pp 365, Interweave Press (2000). As a group, Mentha longifolia is characterized by its unique essential oil chemistry and taxonomic features. For example, the hairy leaf characteristic has been cited as the reason for its common name of Silver or Horse Mint. Horse Mint has been approved by the Japanese Ministry of Health for use as a flavoring in Japanese food products. Japan External Trade Organization, Specifications and Standards for Foods, Food Additives, etc., Under the Food Sanitation Act, Abstracts 2008 (January 2009). However, there are no available records documenting any global production of M. longifolia as a formalized agricultural crop. Further, there are no available records indicating its use in commercial applications.

As a member of the Mentha genus, M. longifolia is a perennial plant with a growing cycle typical of other Mentha ssp. In the northwest growing regions of the United States, planting begins in the spring with the harvest occurring in late summer. For mature fields, in selected areas like the State of Washington, a double cut option exists for this plant. Oil recovery is again typical of standard mint operations. Once harvested, the hay is allowed to dry in the field for one to two days. The hay is then collected, cropped and subjected to a steam distillation in order to recover the oil. The crude oil is dewatered and standardized for future use as a direct flavor additive or reprocessed for other flavor applications.

### SUMMARY

In one aspect, the invention is directed to oral compositions comprising an effective amount of an essential oil sufficient to deliver a physical warming sensation within an oral cavity, wherein the essential oil is derived from Mentha longifolia.

In a further aspect, the invention is directed to oral compositions comprising an effective amount of an essential oil to deliver a physical warming sensation within an oral cavity, wherein the essential oil contains combined levels of cis & trans-Piperitone Oxide greater 35% and levels of Piperitenone Oxide greater than 15%.

In another aspect, the invention is directed to a method for imparting a physical warming sensation within an oral cavity comprising administering an effective amount of an oral composition comprising an essential oil, wherein the essential oil contains combined levels of cis & trans-Piperitone Oxide greater 35% and levels of Piperitenone Oxide greater than 15%.

### BRIEF DESCRIPTION OF THE DRAWINGS

FIG. 1 shows a gas chromatogram of the M. longifolia ssp. hymalaiensis rectified essential oil.

FIG. 2 shows the sensory profile characteristics of M. longifolia ssp. hymalaiensis rectified essential oil.

### DETAILED DESCRIPTION

Both the natural oil as it is isolated directly from the plant or a rectified (redistilled) isolate of the natural oil are shown to possess unique organoleptic characteristics when an effective amount is used as a primary flavoring or as an additive in flavor compositions for chewing gum, confection and oral care applications. An effective amount of the essential oil is an amount capable of imparting a physical warming sensation to the oral cavity when administered in a suitable oral composition. The effective amount may range among various formulations, and may be determined by one of ordinary skill in the art with undue experimentation. The specific examples of the detail description further provide various embodiments of oral compositions containing an effective amount of the essential oil for imparting a physical warming sensation.

As one example, a partially rectified oil was prepared by removing a 10% head or top cut by vacuum distillation at 25 mm Hg. After the 10% head cut has been removed, the remaining 90% of the essential oil or rectified oil is used directly as the flavoring material.

In order to better understand various exemplary embodiments, reference is made to the accompanying drawings, wherein:

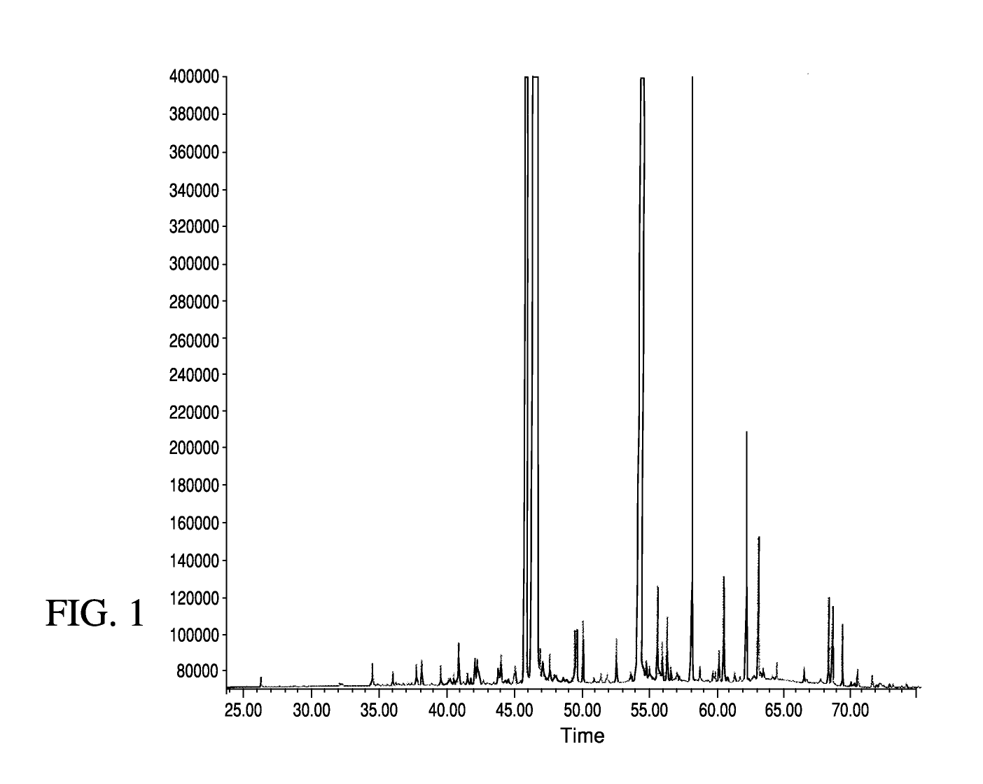

FIG. 1 shows a gas chromatogram of the M. longifolia ssp. hymalaiensis rectified essential oil used as the primary flavoring for the examples provided below. Using a standard Gas Chromatographic/Mass Spectral procedure (GC/MS), the analytical results obtained for the M. longifolia rectified essential oil have been presented in Table 1.

TABLE 1
Mentha longifolia ssp. hymalaiensis Redistilled. Analytical Results

| Peak No, | Ingredient | Amount (%) |
|---|---|---|
| 1 | Linalool | 0.11 |
| 2 | l-Carvone | 13.91 |
| 3 | cis-Piperitone Oxide | 48.62 |
| 4 | trans-Piperitone Oxide | 1.47 |
| 5 | Thymol | 0.26 |
| 6 | 4-Hydroxypiperitone | 0.30 |
| 7 | Piperitenone | 0.22 |
| 8 | Piperitenone Oxide | 20.67 |
| 9 | beta-Bourbonene | 0.46 |
| 10 | cis-Jasmone | 0.30 |
| 11 | beta-Caryophyllene | 3.74 |
| 12 | alpha-Humulene | 0.12 |
| 13 | Germacrene D | 1.20 |
| 14 | delta-Cadinene | 0.98 |
| 15 | Spathulenol | 0.44 |
| 16 | Caryophyllene Oxide | 0.47 |
| 17 | Viridiflorol | 0.31 |
| | Others | 6.42 |
| | Total | 100.00 |

Referencing Table 1 and comparing these data to published data for M. piperita, M. canadensis, M. spicata and M. gracilis, the composition of M. longifolia ssp. hymalaiensis essential oil is unique. M. piperita and M. canadensis essential oils are characterized by their high concentrations of Menthol (4 isomers) and Menthone (2 isomers) which typically account for over sixty five (65) percent of the entire essential oil. In the case of M. spicata and M. gracilis, the concentration of l-Carvone and related chemicals is in excess of sixty (60) percent of the essential oil. M. piperita, M. canadensis, M. spicata and M. gracilis are the only essential oils from the Mentha genus that enjoy a commercial presence in the market and, in all cases, the Piperitone and Piperitenone Oxides would be present only at trace levels in these essential oils.

In an embodiment, the oral compositions are characterized by an essential oil having the combined total of cis and trans-Piperitone Oxide levels of greater than about 20%.

In another embodiment, the oral compositions are characterized by an essential oil having a cis-Piperitone Oxide level of greater than about 20%. In yet another embodiment, the oral compositions are characterized by an essential oil having a trans-Piperitone Oxide level of greater than about 1%.

In another embodiment, the oral compositions are characterized by an essential oil having a Piperitenone Oxide level less than about 30%.

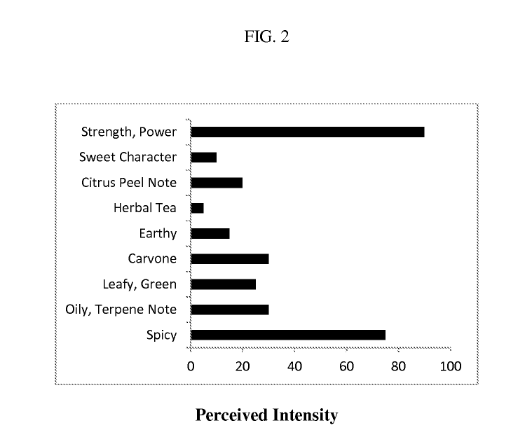

FIG. 2 shows a summary of key sensory attributes deemed important to the overall sensory profile of the oil. The specified organoleptic characteristics were determined using a standard magnitude estimation technique by a trained sensory panel. Intensity is a subjective measurement determined by an Expert Sensory Panel presented as an average from consolidated inputs.

Importantly, the essential oil also delivers a physical warming sensation within the oral cavity. This property is regarded as a very distinctive organoleptic feature and quite different from sensory profiles typical of the commercially available essential oils from the Mentha genus. Beyond the trigeminal physical warming effect, in sensory terms, essential oil exhibits a spicy flavor character.

In addition to being isolated and purified from M. longifolia, the essential oil composition may be prepared using synthetic methods.

In a number of preferred embodiments, M. longifolia essential oil is incorporated into oral compositions as a flavoring in chewing gum, confections, and in oral care applications, such as toothpaste and mouthwash products. Manufacture of chewing gums and confections such as pressed tablets are well known and are taught in E. B. Jackson, Ed. “Sugar Confectionery Manufacture”, 2nd edition, Blackie Academic & Professional Press, Glasgow UK, (1990), at pages 259 and 236 respectively, and in R. Lee and E. B. Jackson, Eds. “Sugar Confectionery and Chocolate Manufacture”, Chapman and Hall, UK, (1992), at pages 332 and 286 respectively. The essential oil can be used directly in its natural (unrectified) or rectified forms as the sole flavor additive or used as an additive within a flavor composition. The oil and/or flavor composition can be delivered in oil or water soluble forms. The flavor system can also be encapsulated or plated where essential or rectified oil is contained within a solid matrix (encapsulated) or simply coated on a solid matrix (plated). The delivery of the flavor can be achieved through water solubility, mechanical shear or thermal release through a selection of the solid matrix or carrier ingredient(s). For selected product applications, delivery can also be achieved through the use of a flavor emulsion.

Various mouthwash products are known to those of ordinary skill in the art. A full treatment of mouthwash history and technology is presented by Rosenthal in Balsam et al. Cosmetics Science and Technology, John Wiley and Sons, 2nd. edition, 1972, Chapter 14 inclusive, which is incorporated herein by reference.

The oral compositions may further include carriers, colorants, additives, additional flavorings, and other functional ingredients as are known in the art. Such functional ingredients include, but are not limited to, sweeteners, flavors, preservatives, dyes, antioxidants, flow agents, thickeners, and emollients.

Carriers include any solid or liquid diluent suitable for oral delivery of an effective amount of the essential oil. For example, for a chewing gum, the carrier may include a gum base such as chicle, jelutong, gutta percha, crown gum, synthetic elastomers such as butadiene-styrene copolymer, isobutylene isoprene copolymer, polyethylene, polyisobutylene and polyvinylacetate, mixtures thereof, and the like. Plasticizers or softeners such as lanolin, stearic acid, sodium stearate, potassium stearate, glyceryl triacetate, glycerin and the like may also be incorporated into the gum base to obtain desirable texture and consistency properties. Generally, the chewing gum may contain any food additive such as those described in “Chemicals Used In Food Processing”, publication 1274, pages 63-258, by the National Academy of Sciences, may be used.

The preparation of confectionery formulations is historically well known. Soft candy confectionery includes fondants, caramels, toffees, fudge, marshmallows and nougats and the like and may also include jams and jellies. A general discussion of the composition and preparation of such confections may be found in E. B. Jackson, Ed. “Sugar Confectionery Manufacture”, 2nd edition, Blackie Academic & Professional Press, Glasgow UK, (1990), at pages 170-235.

### Example 1

### Chewing Gum. Sugar, Sugar-Free, Novelty, Bubblegum and Compressed Chewing Gum Base Applications

Using different shapes and formats including but not limited to stick, pellets (coated and uncoated), layered, slab, center-filled and deposited gum, the preferred essential oil can be added at, but not limited to, a concentration of about 0.1% to about 1.0%, by weight of the confection, for use as the sole flavoring or in a flavor composition. The essential oil may also be combined with colorants, additives for general wellness and other functional ingredients.

### Example 2

### Candy, Hard. Sugar, Sugar-Free, Toffee and Hard-Boiled Candy Applications

Using different pieces and specialty forms including but not limited to layered formats, lozenges and center-filled products, the preferred essential oil can be added at, but not limited to, a concentration of about 0.05% to about 0.3%, by weight of the confection, for use as the sole flavoring or in a flavor composition. The essential oil or flavor composition may also be combined with colorants, additives for general wellness and other functional ingredients.

### Example 3

### Candy, Soft. Sugar, Sugar-Free, Chews, Caramels, Gelatin-Based Jellies, Starch-Based Jellies, Pectin-Based Jellies, Agar-Based Jellies, Konjac-Based Jellies, Jelly Beans, Starch Candy, Nougats, Nougatine, Toffee, Taffy, Marshmallow, Fondant, Fudge, Marzipan, Compound Coatings, Carob Coatings, Gum Drops, Licorice and Fruit Snack Applications

Using different pieces and specialty forms including but not limited to layered formats and center-filled products, the preferred essential oil can be added at, but not limited to, a concentration of about 0.05% to about 0.1%%, by weight of the confection, for use as the sole flavoring or in a flavor composition. The essential oil or flavor composition may also be combined with colorants, additives for general wellness and other functional ingredients.

### Example 4

### Candy, Compressed Tablets. Sugar, Sugar-Free

Using different pieces and specialty forms including but not limited to layered formats and selected coating, the preferred essential oil can be added at, but not limited to, a concentration of about 0.05% to about 0.1%%, by weight of the confection, for use as the sole flavoring or in a flavor composition. The essential oil or flavor composition may also be combined with colorants, additives for general wellness and other functional ingredients.

### Example 5

### Candy, Powders and Dusts. Sugar, Sugar-Free, Dry Mix or Agglomerated Powder Blends

The preferred essential oil can be added at, but not limited to, a concentration of about 0.1% to about 0.5%%, by weight of the confection, for use as the sole flavoring or in a flavor composition. The essential oil or flavor composition may also be combined with colorants, additives for general wellness and other functional ingredients.

### Example 6

### Candy, Other. Edible Films, Lollipops, Nut Brittles, Pastilles, Pralines, Nonpareils, Dragees, Sugared Nuts, Comfits and Aniseed Balls

The preferred essential oil can be added at, but not limited to, a concentration of about 0.05% to about 0.3%, by weight of the confection, for use as the sole flavoring or in a flavor composition. The essential oil or flavor composition may also be combined with colorants, additives for general wellness and other functional ingredients.

### Example 7

### Chocolate and Related Products. Chocolate and Confectioner's Coatings, Milk, White and Dark Chocolate

Using bars and molded pieces with or without inclusions and enrobed centers involving different textures, the preferred essential oil can be added at, but not limited to, a concentration of about 0.2% to about 0.8%%, by weight of the confection, for use as the sole flavoring or in a flavor composition. The essential oil or flavor composition may also be combined with colorants, additives for general wellness and other functional ingredients.

### Example 8

### Oral Care, Toothpaste

The preferred essential oil can be added at, but not limited to, a concentration of about 0.2% to about 0.5%%, by weight of the toothpaste, for use as the sole flavoring or in a flavor composition. The essential oil or flavor composition may also be combined with colorants, additives for general wellness and other functional ingredients.

### Example 9

### Oral Care, Mouthwash

The preferred essential oil can be added at, but not limited to, a concentration of about 0.05% to about 0.2%%, by weight of the mouthwash, for use as the sole flavoring or in a flavor composition. The essential oil or flavor composition may also be combined with colorants, additives for general wellness and other functional ingredients.

### Example 10

### Beverage, Ready-To-Drink

The preferred essential oil can be added at, but not limited to, a concentration of about 0.05% to about 0.2%%, by weight of the beverage, for use as the sole flavoring or in a flavor composition. The essential oil or flavor composition may also be combined with colorants, additives for general wellness and other functional ingredients.

### Example 11

### Beverage, Liquid, Syrup Concentrate

The preferred essential oil can be added at, but not limited to, a concentration of about 0.15% to about 0.9%%, by weight of the concentrate, for use as the sole flavoring or in a flavor composition. The essential oil or flavor composition may also be combined with colorants, additives for general wellness and other functional ingredients.

### Example 12

### Beverage, Powder

The preferred essential oil can be added at, but not limited to, a concentration of about 0.1% to about 0.5%%, by weight of the powder, for use as the sole flavoring or in a flavor composition. The essential oil or flavor composition may also be combined with colorants, additives for general wellness and other functional ingredients.

### Example 13

### Food Preparations

The preferred essential oil can be added at, but not limited to, a concentration of about 0.2% to about 0.75%%, by weight of the food preparation, for use as the sole flavoring or in a flavor composition. The essential oil or flavor composition may also be combined with colorants, additives for general wellness and other functional ingredients.

### Example 14

### Baked Goods

The preferred essential oil can be added at, but not limited to, a concentration of about 0.3% to about 1.0%%, by weight of the baked good, for use as the sole flavoring or in a flavor composition. The essential oil or flavor composition may also be combined with colorants, additives for general wellness and other functional ingredients.

Although the various exemplary embodiments have been described in detail with particular reference to certain exemplary aspects thereof, it should be understood that the invention is capable of other embodiments and its details are capable of modifications in various respects. As is readily apparent to those skilled in the art, variations and modifications can be affected while remaining within the spirit and scope of the invention. Accordingly, the foregoing disclosure, description, and figures are for illustrative purposes only and do not in any way limit the invention, which is defined only by the claims.

What is claimed is:

1. An oral composition comprising:
an effective amount of an essential oil effective to deliver a physical warming sensation within an oral cavity, wherein the essential oil is derived from Mentha longifolia; and
a carrier.

2. The oral composition according to claim 1, wherein the Mentha longifolia is Mentha longifolia ssp. hymalaiensis.

3. The oral composition according to claim 1, wherein the essential oil is rectified.

4. The oral composition according to claim 1, wherein the essential oil contains greater than 15% Piperitenone Oxide.

5. The oral composition according to claim 1, wherein the essential oil contains greater than 20% of cis-Piperitone Oxide and 1% of trans-Piperitone Oxide.

6. The oral composition according to claim 1, wherein the essential oil is present at a concentration of 0.02% to 1.0% by weight of the composition.

7. The oral composition according to claim 1, wherein the oral composition is in the form of a chewing gum, a hard candy, a soft candy, a candy powder, a candy dust, a chocolate product, a toothpaste, a mouthwash, a rinse, or a beverage.

8. An oral composition comprising:
an effective amount of an essential oil effective to deliver a physical warming sensation within an oral cavity, wherein the essential oil contains greater than 15% Piperitenone Oxide; and
a carrier.

9. The oral composition according to claim 8, wherein the essential oil is rectified.

10. The oral composition according to claim 8, wherein the essential oil is derived from Mentha longifolia.

11. The oral composition according to claim 10, wherein the Mentha longifolia is Mentha longifolia ssp. hymalaiensis.

12. The oral composition according to claim 10, wherein the essential oil contains less than 30% Piperitenone Oxide.

13. The oral composition according to claim 8, wherein the essential oil is present at a concentration of 0.02% to 1.0% by weight of the composition.

14. The oral composition according to claim 8, wherein the oral composition is in the form of a chewing gum, a hard candy, a soft candy, a candy powder, a candy dust, a chocolate product, a toothpaste, a mouthwash, a rinse, or a beverage.

15. A method for imparting a physical warming sensation within an oral cavity comprising:
administering an effective amount of an oral composition comprising, an essential oil, wherein the essential oil contains greater than 15% Piperitenone Oxide; and
a carrier.

16. The method according to claim 15, wherein the essential oil is derived from M. longifolia.

17. The method according to claim 16, wherein the Mentha longifolia is Mentha longifolia ssp. hymalaiensis.

18. The method according to claim 15, wherein the essential oil is rectified.

19. The method according to claim 15, wherein the essential oil is present at a concentration of 0.02% to 1.0% by weight of the composition.

20. The method according to claim 15, wherein the oral composition is in the form of a chewing gum, a hard candy, a soft candy, a candy powder, a candy dust, a chocolate product, a toothpaste, a mouthwash, a rinse, or a beverage.
